(19) United States
(12) Patent Application Publication
(10) Pub. No.: US 2019/0159954 A1
(43) Pub. Date: May 30, 2019

(54) BIOMECHANICAL ASSISTIVE DEVICE FOR COLLECTING CLINICAL DATA

(71) Applicant: STEERING SOLUTIONS IP HOLDING CORPORATION, Saginaw, MI (US)

(72) Inventors: Muzaffer Y. Ozsecen, Saginaw, MI (US); Owen K. Tosh, Saginaw, MI (US)

(21) Appl. No.: 16/202,680

(22) Filed: Nov. 28, 2018

Related U.S. Application Data
(60) Provisional application No. 62/591,366, filed on Nov. 28, 2017.

(51) Int. Cl. A61H 3/00; G16H 40/63; A63B 21/00; A63B 24/00; A61H 1/02

(52) U.S. Cl. CPC A61H 3/00; A61H 2201/5084; A63B 21/0004; A63B 21/00181; A63B 21/4011; A63B 24/0062; A61H 1/0244; A63B 2024/0071; A63B 2220/22; A63B 2220/62; A63B 2220/30; A61H 2205/088; A61H 2201/1215; A61H 2201/165; A61H 2201/5061; A61H 2201/5064; A61H 2201/5069; A61H 2201/5076; G16H 40/63

(57) ABSTRACT

One general aspect of technical solutions described herein includes a biomechanical assistive device that includes one or more sensors, a back-drivable motor system, and a controller. The controller, when the motor system is inactive, records measurements from the one or more sensors for user motion pattern analysis during a user activity being performed by a user. The controller, when the motor system is active, records the measurements from the one or more sensors, and generates an assist torque to assist the user to perform the user activity.

### CROSS-REFERENCES TO RELATED APPLICATIONS

This patent application claims priority to U.S. Provisional Patent Application Ser. No. 62/591,366, filed Nov. 28, 2017, which is incorporated herein by reference in its entirety.

### BACKGROUND

Exoskeletons are devices that can amplify a person's natural ability and improve their quality of life. In one or more examples, exoskeleton devices facilitate overcoming physical human limitations by amplifying human strength, endurance, and mobility potential. The exoskeleton devices are thus biomechanical assistive devices that may be worn by a user, for example worn in association with a joint in the body, to amplify or improve the functioning of that joint.

Exoskeleton devices can be classified as either passive or powered devices. A passive device typically cannot generate and deliver energy external to the user, rather a passive device helps the user employ his own muscle power more effectively. Passive devices can include springs, and can store potential energy and deliver it in addition to the human motion. One example of exoskeleton-based passive assist is passive gravity support where the exoskeleton supports part of the user's weight. However, the exoskeleton cannot contribute to raise the user's center of gravity, for example when getting up from a chair.

A powered exoskeleton device on the other hand generates and supplies energy to the user through external means (i.e. electrical, hydraulic, etc.), in one or more examples, in a continuous way, to help the user to elevate the center of mass of the body at one point or another by generating torque, for example using one or more actuators. The biomechanical assistive devices that are described herein are powered exoskeleton devices.

For operation of the assistive devices, the devices have to provide the appropriate amount of torque to assist with the user's activity, one way of providing such assist is done by detecting the user's current activity (ex. walking, standing, sitting). Typically, the assistive devices require direct user input, or are very slow to recognize activities automatically. Accordingly, there is a need for the assistive devices to automatically recognize user activity within a predetermined duration threshold.

### SUMMARY

One general aspect includes a biomechanical assistive device that includes one or more sensors, a back-drivable motor system, and a controller. The controller, when the motor system is inactive, records measurements from the one or more sensors for user motion pattern analysis during a user activity being performed by a user. The controller, when the motor system is active, records the measurements from the one or more sensors, and generates an assist torque to assist the user to perform the user activity.

According to another aspect, a method for operating a biomechanical assistive device includes, based on a motor system of the biomechanical assistive device being inactive, recording kinematic parameters for user motion pattern analysis, the kinematic parameters computed using measurements from one or more sensors during a user activity being performed by a user wearing the biomechanical assistive device. The method further includes, based on the motor system being active, recording the kinematics parameters, and generating an assist torque using an actuator to assist the user to perform the user activity.

According to one or more embodiments, a computer program product for operating a biomechanical assistive device includes computer readable storage medium with computer executable instructions therein, the computer executable instructions cause a processing circuit to perform a method. The method includes, based on a motor system of the biomechanical assistive device being inactive, recording kinematic parameters for user motion pattern analysis, the kinematic parameters computed using measurements from one or more sensors during a user activity being performed by a user wearing the biomechanical assistive device. The method further includes, based on the motor system being active, recording the kinematics parameters, and generating an assist torque using an actuator to assist the user to perform the user activity.

These and other advantages and features will become more apparent from the following description taken in conjunction with the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 is a perspective view of an exemplary adjustable biomechanical assist device according to one or more embodiments;

FIG. 2 depicts an example controller according to one or more embodiments;

FIG. 3 depicts a block diagram of the biomechanical assistive device in operation according to one or more embodiments; and

FIG. 4 depicts an example workflow for the user motion pattern data being captured according to one or more embodiments.

### DETAILED DESCRIPTION

An exoskeleton, particularly, an active exoskeleton is a biomechanical assistive device that provides torque assist at a human joint, such as the hip joint. Technical challenges with assistive devices exist with the lack of recording key motion parameters, such as indicators of the performance of a user (human). The technical solutions described herein facilitate biomechanical assistive devices, such as exoskeleton devices, to identify and record data for the key motion parameters of a user activity in both passive (no augmentation) and active (motion augmentation) modes. In the active mode a motor system of the exoskeleton generates and provides an assist torque to the user to complete one or more activities. In the passive mode the motor system that generates the assist torque is switched OFF, and accordingly, the assist torque is not being provided to the user.

Technical solutions are described for addressing such technical challenges with assistive devices and to facilitate biomechanical assistive devices to identify and record data for the key motion parameters of the user activity in both passive (no augmentation) and active (motion augmentation) modes.

Further, technical challenges exist for the assistive device to recognize the user's current activity (walking, standing, sitting, etc.) and further identify and record key motion parameters (indicators of the performance of the user) so based on the recognized activity. It should be noted that the biomechanical assistive device also determines an appropriate amount of assist torque that is to be generated and provided to the joint/user based on such automatic recognition of the activity. Presently, the assistive devices generate torque and record key motion parameters either based on measurements of the user activity that are collected using wearable devices such as accelerometers, or based on separate stand-alone devices such as cameras/motion detectors. However, the wearable devices typically do not provide precise measurements when the user is performing the user activity while wearing the biomechanical assistive device, and the stand-alone devices add limitations to where the data may be collected.

Further, human motion analysis is challenging to accomplish while the user is “wearing” the biomechanical assistive device, without “using” the biomechanical assistive device. Presently, in one or more examples, users have to remove the biomechanical assistive device to measure such key motion parameters and re-wear the biomechanical assistive device for the data collection. Along with user discomfort, particularly with users that may have a physical handicap, this can cause lengthen the time required for data collection.

It is also difficult to integrate data gathered from different systems, such as the wearable devices and stand-alone devices. For example, different parts of the user's body may be monitored by different types of devices to gather such data, and then integrated after the collection. Accordingly, existing solutions fail to report key motion parameters in a user friendly way using a single device.

In one or more examples, present biomechanical assistive devices collect only the key motion parameter data during user activities when the biomechanical assistive device itself is being actively used, and further the collected data only includes the parameters that the biomechanical assistive device creates or generates. For example in gait training exoskeletons (e.g. EKSOGT™, REWALK™) motion data collected by the biomechanical assistive device is based on a pre-programmed position trajectory as opposed to user's motion.

The technical solutions described herein improve the data collection by using the biomechanical assistive device to produce user performance measurements such as cadence, and other clinical functions and providing motion augmentation by generating the torque assist based on the captured data parameter measurements. The technical solutions described herein address such technical challenges by facilitating the biomechanical assistive device itself to identify and record key motion parameters for the user activity. Generating clinically relevant (user performance) data in parallel to the operation of the assistive device facilitates the estimation, logging, and categorization of user (wearer) activity patterns. These patterns are further analyzed to detect and identify strengths, weaknesses, adherence, and motion habits of the user. Generating and using these patterns facilitates documenting the progress of the user and it can ease a clinician's effort to report clinical outcomes.

The technical solutions described herein address such technical challenges in assistive devices using an actuator that has a back-drive capability in passive mode. A passive mode for the biomechanical assistive device is when the biomechanical assistive device is not actively being used to generate torque. In the passive mode, the user performs the user activity without the assistive device providing any assistive torque, rather the biomechanical assistive device only collects the key motion parameters of the user's actions to perform the activity. In addition, the technical solutions described herein facilitate the assistive device to continue to collect the key motion parameters for the user to recognize activity and measure clinical functions when the biomechanical assistive device is being used in an active mode. The active mode is when the biomechanical assistive device is being used to provide assistance torque to the user to perform the activity. In one or more examples, the assistive device can be switched between the active mode and the passive mode, which in turn, switches a motor system of the assistive device on and/or off. Accordingly, the passive mode can also be considered an inactive mode for the motor system.

In one or more examples, the parameters measured and recorded by the biomechanical assistive device includes gait parameters measured using sensors located on the biomechanical assistive device that is worn by the user. For example, the sensors measure position, speed, acceleration, force, and the like. Using input from the sensors, a controller determines motion patterns for the user, the motion parameters being stored for further analysis.

By measuring the joint kinematics parameters, the technical solutions described herein facilitate the biomechanical assistive device performance to improve over typical solutions, such as pedometers (or other pendants) in measuring step count, estimating step length, cadence, and other gait parameters. Combination of the user performance measurement (Clinical Functions) and generation and utilization of torque assist based on identifying the user activity automatically and without user input facilitates the biomechanical assistive device to be a useful tool both in clinical and home use. Further, a controller architecture that provides a combination of such features in a single device is used to automatically recognize user activity and track user progress and to generate reports regarding the user progress. Various gait parameters, combined with user specific data is stored to later formulate a database to study disorders, utilizing big data analysis techniques, such as machine learning, neural networks, and the like. Further, the captured information provides statistics to clinicans for designing further technical solutions and hypotheses.

The technical solutions described herein use embodiments directed to a hip-joint assistive device, however, it will be appreciated that the technical solutions can be implemented in biomechanical assistive devices used at other joints in a body.

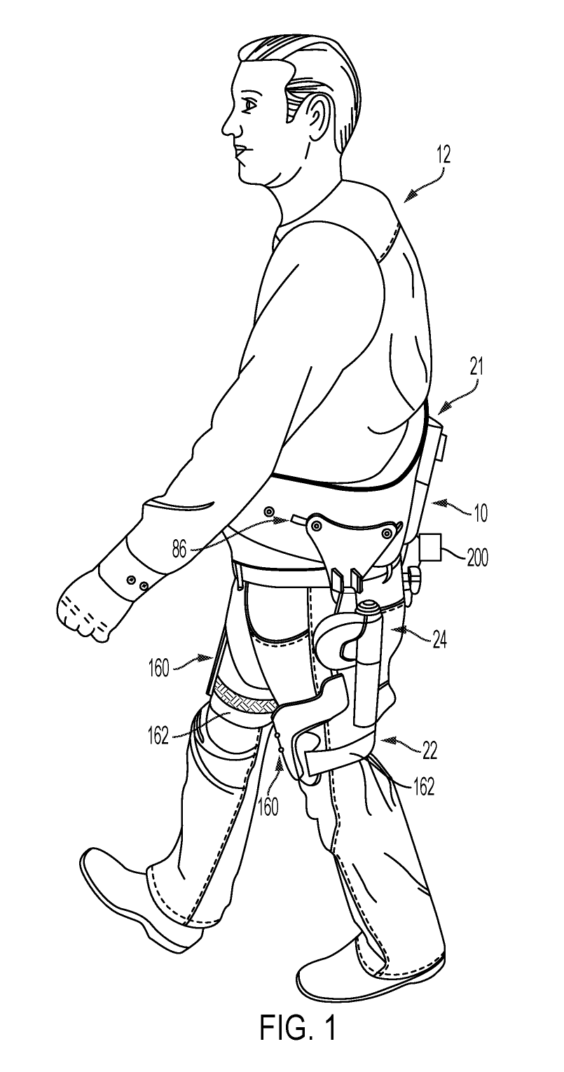

Referring now to the figures, FIG. 1 is a perspective view of an exemplary adjustable biomechanical assist device 10 according to one or more embodiments. Here, an environmental view of a powered assistive device 10 that is attachable to a user 12 is shown. The powered assistive device 10 is wearable by the user 12 to aid the user 12 in performing various movements, tasks, or to reduce the user's energy consumption during various movements. The powered assistive device 10 is mechanically grounded to a portion of the user 12 to aid in the transfer of torque by the powered assistive device 10 to the user 12. The powered assistive device 10 includes a lumbar support apparatus 21, at least one leg support 22, and an actuator 24.

The lumbar support apparatus 21 is configured as a torso brace that interfaces with the user 12. The lumbar support apparatus 21 is disposed about a user's waist proximate a user's hip region. The lumbar support apparatus 21 is configured to adjust overall human-exoskeleton interface stiffness through the use of various lumbar support types. The various lumbar support types permit the user 12 to adjust for comfort and load or torque transfer efficiency from the powered assistive device 10 to the user 12. The assistive device 10 further includes a controller 200. It should be noted that the depicted assistive device 10 is an example and that the technical solutions described herein are applicable to other types of biomechanical assistive devices too.

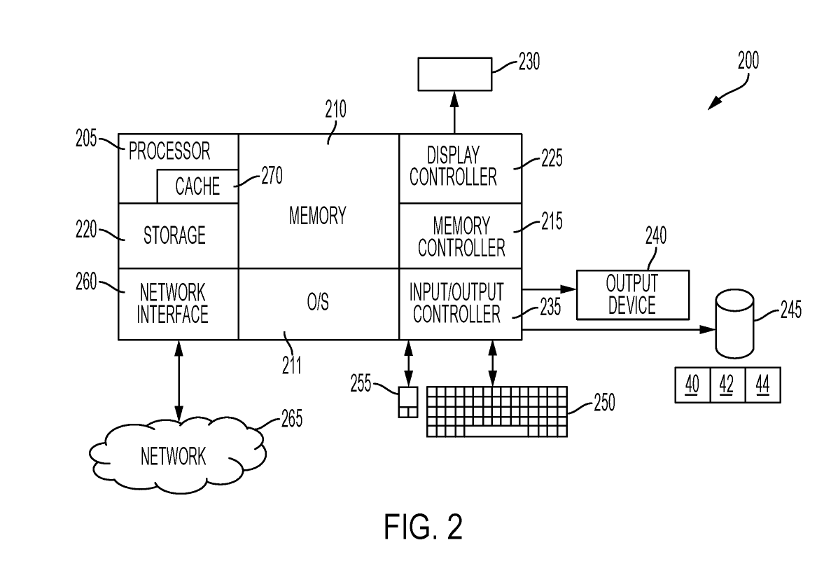

FIG. 2 depicts an example controller 200 according to one or more embodiments. The system 200 includes, among other components, a processor 205, memory 210 coupled to a memory controller 215, and one or more input devices 245 and/or output devices 240, such as peripheral or control devices, that are communicatively coupled via a local I/O controller 235. These devices 240 and 245 may include, for example, battery sensors, position sensors (gyroscope 40, accelerometer 42, GPS 44), indicator/identification lights and the like. Input devices such as a conventional keyboard 250 and mouse 255 may be coupled to the I/O controller 235. The I/O controller 235 may be, for example, one or more buses or other wired or wireless connections, as are known in the art. The I/O controller 235 may have additional elements, which are omitted for simplicity, such as controllers, buffers (caches), drivers, repeaters, and receivers, to enable communications.

The I/O devices 240, 245 may further include devices that communicate both inputs and outputs, for instance disk and tape storage, a network interface card (NIC) or modulator/demodulator (for accessing other files, devices, systems, or a network), a radio frequency (RF) or other transceiver, a telephonic interface, a bridge, a router, and the like.

The processor 205 is a hardware device for executing hardware instructions or software, particularly those stored in memory 210. The processor 205 may be a custom made or commercially available processor, a central processing unit (CPU), an auxiliary processor among several processors associated with the system 200, a semiconductor based microprocessor (in the form of a microchip or chip set), a macroprocessor, or other device for executing instructions. The processor 205 includes a cache 270, which may include, but is not limited to, an instruction cache to speed up executable instruction fetch, a data cache to speed up data fetch and store, and a translation lookaside buffer (TLB) used to speed up virtual-to-physical address translation for both executable instructions and data. The cache 270 may be organized as a hierarchy of more cache levels (L1, L2, and so on.).

The memory 210 may include one or combinations of volatile memory elements (for example, random access memory, RAM, such as DRAM, SRAM, SDRAM) and nonvolatile memory elements (for example, ROM, erasable programmable read only memory (EPROM), electronically erasable programmable read only memory (EEPROM), programmable read only memory (PROM), tape, compact disc read only memory (CD-ROM), disk, diskette, cartridge, cassette or the like). Moreover, the memory 210 may incorporate electronic, magnetic, optical, or other types of storage media. Note that the memory 210 may have a distributed architecture, where various components are situated remote from one another but may be accessed by the processor 205.

The instructions in memory 210 may include one or more separate programs, each of which comprises an ordered listing of executable instructions for implementing logical functions. In the example of FIG. 2, the instructions in the memory 210 include a suitable operating system (OS) 211. The operating system 211 essentially may control the execution of other computer programs and provides scheduling, input-output control, file and data management, memory management, and communication control and related services.

Additional data, including, for example, instructions for the processor 205 or other retrievable information, may be stored in storage 220, which may be a storage device such as a hard disk drive or solid state drive. The stored instructions in memory 210 or in storage 220 may include those enabling the processor to execute one or more aspects of the systems and methods described herein.

The system 200 may further include a display controller 225 coupled to a user interface or display 230. In some embodiments, the display 230 may be an LCD screen. In other embodiments, the display 230 may include a plurality of LED status lights. In some embodiments, the system 200 may further include a network interface 260 for coupling to a network 265. The network 265 may be an IP-based network for communication between the system 200 and an external server, client and the like via a broadband connection. In an embodiment, the network 265 may be a satellite network. The network 265 transmits and receives data between the system 200 and external systems. In some embodiments, the network 265 may be a managed IP network administered by a service provider. The network 265 may be implemented in a wireless fashion, for example, using wireless protocols and technologies, such as WiFi, WiMax, satellite, or any other. The network 265 may also be a packet-switched network such as a local area network, wide area network, metropolitan area network, the Internet, or other similar type of network environment. The network 265 may be a fixed wireless network, a wireless local area network (LAN), a wireless wide area network (WAN) a personal area network (PAN), a virtual private network (VPN), intranet or other suitable network system and may include equipment for receiving and transmitting signals.

In one or more examples, using only two position sensors (one for each hip position), the technical solutions described herein facilitates the assistive device to recognize a new activity of a user with no additional user input and transition to a torque profile for the new activity within the predetermined duration. For example, the assistive device identifies different activities of the user such as sitting, standing, sit-to-stand, stand-to-sit, and walking, and other such activities, and facilitates near real-time transition from one activity (present activity) to another activity (new activity) that the user began without any explicit input from the user identifying the new activity. The technical solutions described herein thus facilitate an intuitive operation of the assistive device for the user, in turn improving the performance of the assistive device.

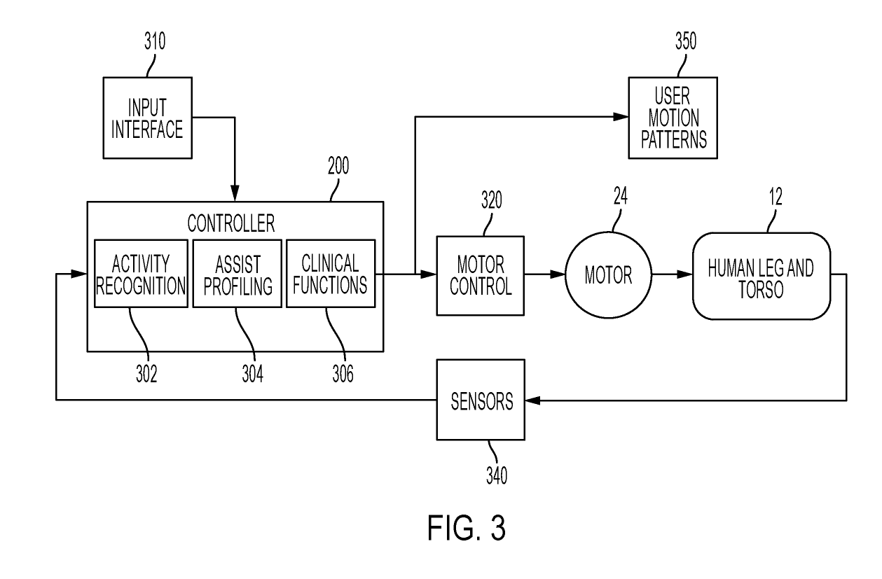

FIG. 3 depicts a block diagram of the biomechanical assistive device in operation according to one or more embodiments. Here, the controller 200 is shown to perform at least three operations of activity recognition 302, assist profiling 304, and clinical operations 306.

The controller 200 performs such operations based on one or more instructions stored in a memory device of the controller 200, and/or based on one or more inputs. The inputs can be received from the user 12 or from a clinician or any other personnel monitoring the user's activities when using the biomechanical assistive device 10. The inputs can be received in a wired or a wireless manner via input interface 310.

The activity recognition 302 facilitates the controller 200 to automatically determine what activity the user 12 is about to perform based on input from one or more sensors 340. The sensors 340 can include position sensors, for example. For example, the biomechanical assistive device 10 operates as a (or using a) finite state machine. In such a case, each activity is considered a ‘state’ of the state machine and determining when to transition from one activity (state) to another is defined by the state machine. A finite state machine is broadly defined as a system with a finite number of discrete states, where each state has criteria to transition to one or more other states of the state machine. The state machine may be operated based on the sensor input, such as position of a hip, leg, or other types of joints of the user 12. For example, the activity recognition 302 identifies different activities of the user 12 such as sitting, standing, sit-to-stand, stand-to-sit, walking, staircase climbing, staircase descent, climbing up a ramp, climbing down a ramp, squatting, lifting and other such activities. The activities can be on an even or an uneven terrain.

The user activity that is detected is used by the assist profiling 304 to determine a torque command to be provided to a motor control system 320. For example, the assist profiling 304 can select a particular torque assist profile based on the detected user activity. The torque assist profile, in one or more examples, can provide a computation of the amount of torque to be generated to assist the user 12 to complete the user activity based on one or more sensor inputs. Further the user activity that is detected is used to determine a motor velocity command to be provided to the motor control system 320. The motor control system 320 uses the input commands to operate the motor (actuator) 24 of the assistive device 10 to generate a corresponding amount of torque and/or displacement of the motor 24 to provide the assist to the user 12.

Further, once the user activity has been detected/identified, the clinical operations 330 capture one or more sensor data to record user motion patterns 350 of the user 12. In one or more examples, the sensor data that is captured is based on the identified user activity because each user activity may be associated with a corresponding set of kinematics parameter measurements to be captured. The clinical operations 330 measures and estimates gait parameters using the sensors 340 located on the assistive device 10 that is worn by the user 12. The sensors 340 can measure position, speed, acceleration, and force, and other such parameters. Using input from the sensors 340 user motion patterns are measured, estimated and logged.

Further yet, the assistive device 10 can capture on or more user-specific data while the user activity is being performed, such as the user height, weight, and other user measurements. In one or more examples, the assistive device 10 stores the captured sensor data corresponding to one or more clinical functions. The captured user motion pattern(s) 350 can include clinical function data that is provided via one or more communication channels. For example, the captured data may be provided for generating one or more reports for the assistive device 10 and/or the user 12. In one or more examples, the captured data is stored in one or more storage devices or memory devices that are part of the assistive device 10 itself. Alternatively, or in addition, the captured data may be provided to one or more external analysis systems.

In one or more examples, the user motion pattern(s) 350 data is continuously captured even when the assistive device 10 is not being used for performing one of the predetermined user activities for which the assistive device 10 provides torque assist. In other words, the clinical operations 330 captures the user motion patterns when the assistive device 10 is in active mode, as well as when the assistive device 10 is in passive/inactive mode. This facilitates the assistive device 10 to capture kinematics parameters for the user 12 in the passive mode, and use the captured kinematics parameters to be further analyzed to generate a torque assist for the user 12 when s/he switches the assistive device 10 to active mode, that is, performing a user activity with the assistive device 10 providing torque assist.

Capturing such kinematics patterns, which is clinically relevant (user performance) data, in parallel to the active mode operation of the biomechanical assistive device 10 allows the estimation, logging, and categorization of wearer activity patterns. These patterns can point out strengths, weaknesses, adherence and motion habits of the user 12. Generating and using these patterns is an important way to document the progress of the user 12 and it can ease the clinician's effort to report clinical outcomes.

The clinical operations 330 can capture the user kinematics data when the assistive device 10 is in the passive mode because of the motor control system 320 and the motor 24 facilitating a back-drivable system. A system is considered back-drivable if a force or torque on its output can move its input. Here, when the assistive device 10 is worn, and is in passive mode, that is the assistive device 10 is not generating an assist torque, the movements of the user 12 causes the one or more mechanical components of the assistive device 10, such as lumbar support 21, the leg support 22, to move. As the one or more mechanical components move, the sensors 340 measure and provide corresponding sensor signals to the controller 200. Such sensor values are also recorded as part of the captured data for the user motion patterns 350.

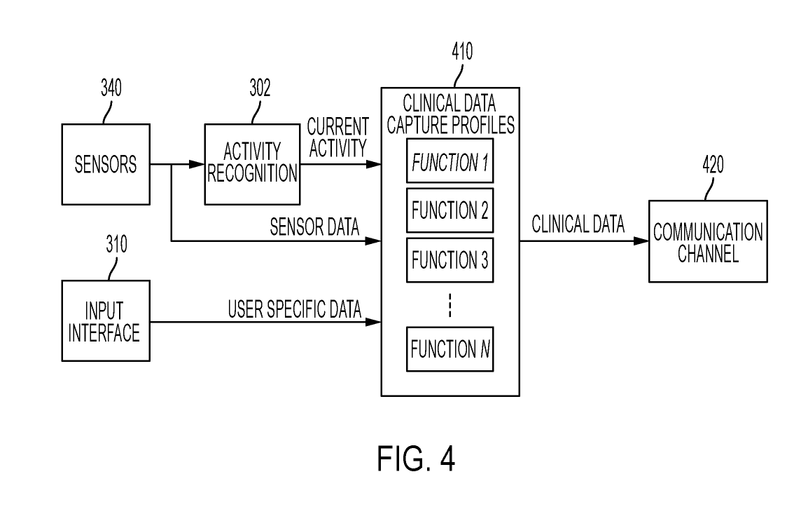

FIG. 4 depicts an example workflow for the user motion pattern data being captured according to one or more embodiments. The data captured using the assistive device 10 can include step angle, step time, step width, stance time, swing time, stride length, stride frequency, stride velocity, stride confidence, cadence(e.g. steps per minute), ground speed, traversed distance, gait autonomy, gait phases, stop duration, route, range of motion and the like. The stride confidence, in one or more examples, is a value (e.g. 0-100%) representing a rate of the assistive device's 10 confidence in calculating the correct stride value. The range of motion is a range of position signal [min max] at certain motion events. For example, normative walking range of motion is: −10 to 40 degrees, i.e. total of 50 degrees-10 degrees of extension (leg going back) 40 degrees of flexion (leg going forward). The range of motion can change based on assist/no assist, user's 12 health condition, and can change from step to step, over time, and the like.

In one or more examples, a clinician, or the user 12, can select one or more of these kinematic parameters to be recorded as part of the data captured for the user motion patterns 350. For example, the selection can be made using the input interface 310 to select from one or more clinical data capture profiles 410. Each of the clinical data capture profile can indicate what type of kinematic parameters are to be captured and recorded for the user 12.

In addition, each of the clinical data capture profiles 410 can include indication of which specific kinematic parameters to capture for particular user activities. For example, when the user 12 is sitting, the step count, and step length may not be recorded and stored. Alternatively, or in addition, in case of a particular user 12, the step length may not be recorded and stored even when the particular user 12 is walking. Accordingly, the identified user activity from the activity recognition 302 is used to determine what activity is being performed and accordingly, the corresponding kinematic parameters from the sensors 340 are recorded.

It should be noted that the user activity detection and the kinematics parameter capture is performed regardless of whether the user 12 is using the assistive device in the active mode or in the passive mode. The back-drivability of the motor 24 and other mechanical components facilitates capturing the kinematics parameters when the assistive device is in the passive mode.

Once the sensor data is captured for the kinematics parameters, the captured data is stored in the assistive device or an external device via a communication channel 420. The communication channel 420 can use a particular protocol, particular encryption, or the like. For example, the communication channel 420 can ensure that the captured data is stored in regulation compliant and secure manner. The data captured corresponding to the one or more clinical data capture profiles is further provided for further analysis and reporting the communication channel 420. In one or more examples, the data may be provided to an external analysis system.

Accordingly, the technical solutions described herein facilitate a single device, the biomechanical assistive device 10 to be used for, first, generating the assist torque for the user activity when the user 12 wears the assistive device 10; and, second, recording clinical data when the user 12 moves while wearing the assistive device 10 in an inactive mode, where the assist torque is not being generated. It should be noted that the clinical data is also recorded in the active mode, where the assist torque is generated. The collected clinical data can be used to analyze user motion patterns and adjustments to be made to one or more settings of the assistive device 10 for the particular user 12. For example, the settings can include an amount of torque to be generated when the user 12 is performing a particular type of user activity. Further, the analysis can result in specific actions to be performed by the user 12, for example, to improve the user's performance when wearing the assistive device 10, or without the assistive device 10.

The technical solutions described herein, by using a single device to do both, the data collection, and torque generation, in addition to saving users' time from changing from one system to another for these functions, improve accuracy of the amount of assist torque that is generated. For example, in existing techniques where the clinical data was collected using a first system, and the assist torque generation was performed by a second, separate system, the effects of the first system had to be compensated for when determining the amount of torque to be generated by the second system. Such compensation was based on a model of the first system. Such compensation, typically, affected the accuracy of the amount of torque generated. Accordingly, the technical solutions described herein provide an improvement to existing biomechanical assistive devices that determine amount of torque to be generated based on user motion pattern analysis.

The technical solutions described herein use embodiments directed to a hip-joint assistive device, however, it will be appreciated that the technical solutions can be implemented in assistive devices used at any other joint, limb, or extremity in a body such as the ankle, knee, or hip joint of a leg or the wrist, elbow, or shoulder joint of an arm. Also, the user can be a human or an animal. Additionally, for ease of explanation, the term “limb” may be used to describe a limb segment (such as a lower leg or an upper arm) attached to a joint of a limb.

It should be noted that although the technical solutions described herein use embodiments in the context of particular biomechanical assistive devices, the technical solutions can be used in other devices that use a state machine, such as in an electric power steering (EPS) systems for signal arbitration (position, torque, speed, etc.), in an EPS for loss of assist mitigation and arbitration. The technical solutions described herein can also be used in an automotive for collision avoidance for autonomous and semi-autonomous vehicles, or for calculating a safest path to pass a vehicle in front. Alternatively, or in addition, the technical solutions described herein are applicable in an EPS, such as a Steer by wire system for initialization process (checking clutch, hand wheel, road wheel sensors etc.), or other diagnostics to be performed. The above is a non-limiting, exemplary list of applications for the technical solutions herein.

While the technical solutions have been described in detail in connection with only a limited number of embodiments, it should be readily understood that the technical solutions are not limited to such disclosed embodiments. Rather, the technical solutions can be modified to incorporate any number of variations, alterations, substitutions or equivalent arrangements not heretofore described, but which are commensurate with the spirit and scope of the technical solutions. Additionally, while various embodiments of the technical solutions have been described, it is to be understood that aspects of the technical solutions may include only some of the described embodiments. Accordingly, the technical solutions are not to be seen as limited by the foregoing description.

What is claimed is:

1. A biomechanical assistive device comprising:
one or more sensors;
a back-drivable motor system; and
a controller configured to:
record measurements from the one or more sensors for user motion pattern analysis during a user activity being performed by a user when the motor system is inactive; and
record the measurements from the one or more sensors, and generate an assist torque to assist the user to perform the user activity when the motor system is active.

2. The biomechanical assistive device of claim 1, wherein the measurements include a measurement from a first sensor from the one or more sensors based on the user activity being a particular type.

3. The biomechanical assistive device of claim 1, wherein the controller is further configured to:
receive a selection of a data capture profile for the user activity;
identify, automatically, that the user activity is being performed; and
record the measurements from a particular subset of the one or more sensors, the particular subset being identified in the data capture profile that is selected.

4. The biomechanical assistive device of claim 1, wherein the user activity is one from a group of user activities comprising: sitting, standing, walking, sit-to-stand transitioning, stand-to-sit transitioning, staircase climbing, staircase descent, climbing up a ramp, climbing down a ramp, squatting, and lifting.

5. The biomechanical assistive device of claim 1, wherein the measurements that are recorded include at least one from a group of measurements comprising step length, step angle, step time, step width, stance time, swing time, stride length, stride frequency, stride velocity, stride confidence, cadence, ground speed, traversed distance, gait autonomy, gait phases, stop duration, route, and range of motion.

6. The biomechanical assistive device of claim 1, wherein the one or more sensors include at least one position sensor.

7. The biomechanical assistive device of claim 1, wherein the motor system is configured to generate assistive torque based on a torque profile that is associated with the user activity being performed by the user.

8. A method for operating a biomechanical assistive device, the method comprising:
recording kinematic parameters for user motion pattern analysis, the kinematic parameters computed using measurements from one or more sensors during a user activity being performed by a user wearing the biomechanical assistive device based on a motor system of the biomechanical assistive device being inactive; and
recording the kinematics parameters, and generating an assist torque using an actuator to assist the user to perform the user activity based on the motor system being active.

9. The method of claim 8, wherein the measurements include a measurement from a first sensor from the one or more sensors based on the user activity being a particular type.

10. The method of claim 8, wherein the method further comprises:
receiving a selection of a data capture profile for the user activity;
identifying, automatically, that the user activity is being performed; and
recording the kinematics parameters based on measurements from a particular subset of the one or more sensors, the particular subset being identified in the data capture profile that is selected.

11. The method of claim 8, wherein the user activity is one from a group of user activities comprising: sitting, standing, walking, sit-to-stand transitioning, and stand-to-sit transitioning, staircase climbing, staircase descent, climbing up a ramp, climbing down a ramp, squatting, and lifting.

12. The method of claim 8, wherein the kinematic parameters that are recorded include at least one from a group of kinematic parameters comprising step length, step angle, step time, step width, stance time, swing time, stride length, stride frequency, stride velocity, stride confidence, cadence, ground speed, traversed distance, gait autonomy, gait phases, stop duration, route, and range of motion.

13. The method of claim 8, wherein the one or more sensors include at least one position sensor.

14. The method of claim 8, wherein the assistive torque is generated based on a torque profile that is associated with the user activity being performed by the user.

15. A computer program product for operating a biomechanical assistive device, the computer program product comprising computer readable storage medium with computer executable instructions therein, the computer executable instructions cause a processing circuit to perform a method comprising:
recording kinematic parameters for user motion pattern analysis, the kinematic parameters computed using measurements from one or more sensors during a user activity being performed by a user wearing the biomechanical assistive device based on a motor system of the biomechanical assistive device being inactive; and
recording the kinematics parameters, and generating an assist torque using an actuator to assist the user to perform the user activity based on the motor system being active.

16. The computer program product of claim 15, wherein the measurements include a measurement from a first sensor from the one or more sensors based on the user activity being a particular type.

17. The computer program product of claim 15, wherein the method further comprises:
receiving a selection of a data capture profile for the user activity;
identifying, automatically, that the user activity is being performed; and
recording the kinematics parameters based on measurements from a particular subset of the one or more sensors, the particular subset being identified in the data capture profile that is selected.

18. The computer program product of claim 15, wherein the user activity is one from a group of user activities comprising: sitting, standing, walking, sit-to-stand transitioning, and stand-to-sit transitioning, staircase climbing, staircase descent, climbing up a ramp, climbing down a ramp, squatting, and lifting.

19. The computer program product of claim 15, wherein the kinematic parameters that are recorded include at least one from a group of kinematic parameters comprising step length, step angle, step time, step width, stance time, swing time, stride length, stride frequency, stride velocity, stride confidence, cadence, ground speed, traversed distance, gait autonomy, gait phases, stop duration, route, and range of motion.

20. The computer program product of claim 15, wherein the assistive torque is generated based on a torque profile that is associated with the user activity being performed by the user.
